(19) United States
(12) Patent Application Publication
(10) Pub. No.: US 2013/0053946 A1
(43) Pub. Date: Feb. 28, 2013

(54) BIOABSORBABLE POLYMER STENT WITH METAL STIFFENERS

(71) Applicant: Jonathan S. Stinson, Plymouth, MN (US)

(72) Inventor: Jonathan S. Stinson, Plymouth, MN (US)

(73) Assignee: BOSTON SCIENTIFIC SCIMED, INC., Maple Grove, MN (US)

(21) Appl. No.: 13/240,879

(22) Filed: Sep. 22, 2011

Related U.S. Application Data
(60) Provisional application No. 61/529,086, filed on Aug. 30, 2011.

(51) Int. Cl. A61F 2/82; A61F 2/86

(52) U.S. Cl. 623/1.15; 264/279.1

(57) ABSTRACT

A composite stent comprises an expandable framework made from a bioabsorbable polymer and a plurality of metallic structures disposed on, adhered to or force fit into openings of the expandable framework. Each opening has a perimeter defined by a plurality of struts of the expandable framework. Each strut has a width and a thickness. At least one first metallic structure is disposed along at least a portion of the perimeter of at least one of the openings. Methods for manufacturing such a composite stent are provided herein.

### CROSS REFERENCE TO RELATED APPLICATIONS

This application claims the benefit of U.S. Provisional Application No. 61/529,086, entitled, “Bioabsorbable Polymer Stent with Metal Stiffeners,” by Jonathan S. Stinson, and filed on Aug. 30, 2011, the entire contents of which being incorporated herein by reference.

### BACKGROUND OF THE INVENTION

A stent is a medical device that is introduced into a body lumen and is well known in the art. A stent is typically delivered in an unexpanded state to a desired location in a bodily lumen and then expanded by an internal radial force.

Stents, grafts, stent-grafts, vena cava filters, expandable frameworks, and similar implantable medical devices, are radially expandable endoprostheses, which are typically intravascular implants capable of being implanted transluminally and enlarged radially after being introduced percutaneously. Stents may be implanted in a variety of bodily lumens or vessels such as within the vascular system, urinary tracts, bile ducts, fallopian tubes, coronary vessels, secondary vessels, etc. Stents can be balloon-expandable, self-expanding or a combination of self-expanding and balloon-expandable (or “hybrid expandable”).

Stents are commonly manufactured from either metal or polymer tubes of a single material, often by laser or chemical machining. Since stents are commonly made from tubing that is composed of one material, the stent mechanical properties are dependent upon the properties of that material. Stents made of metal typically have relatively high strength, stiffness, and radiopacity and less elastic recoil upon expansion relative to stents made of polymer. This is because metals tend to have a higher Young's modulus of elasticity, higher yield strength, higher work hardening rate, and higher density than polymers. Polymer stents typically have more axial and radial flexibility than metal stents with the same wall thickness due to the polymer's much lower modulus of elasticity.

The disparity in mechanical properties between stents made from polymers and stents made from metals is particularly apparent with stents manufactured from bioabsorbable polymers, which desirably degrade in the body into naturally occurring chemical species that are readily metabolized or excreted, rather than leaving minerals and metal corrosion products behind. The mechanical properties of bioabsorbable polymer stents are far from their metal counterparts, requiring significant compromises in design in order to close the gap in mechanical properties. For example, in order to reach the radial strength and stiffness of metal stents, polymer stents need to have a wall thickness that is at least 30% more than metal stents, in some cases 100% more or even greater than 200% of the thickness of a comparable metal stent. This undesirably increases the profile of the polymer stent such that it occupies more of the vessel luminal area, thus reducing the volume of fluid flow in the stented lumen.

It is desirable, therefore, to have a bioabsorbable stent that combines the desirable material properties of bioabsorbable polymer stents (such as increased flexibility) with the desirable material properties of metal stents (such as radial strength and stiffness) to reduce the overall profile of the stent delivery system device.

### BRIEF SUMMARY

In at least one embodiment, a composite stent comprises an expandable framework and a plurality of metallic structures disposed on, adhered to or force fit into the expandable framework. The expandable framework comprises a bioabsorbable polymer and defines a plurality of openings, where each opening has a perimeter defined by a plurality of struts of the expandable framework. Each strut has a width and a thickness. At least one first metallic structure is disposed along at least a portion of the perimeter of at least one of the openings. The first metallic structure has a width and a thickness. In at least one embodiment, the thickness of the at least one first metallic structure is less than the thickness of the strut. In at least one embodiment, the width of the at least one first metallic structure is less than the width of the strut. In some embodiments, the metallic structures are struts or ring-like structures. Methods for manufacturing such a composite stent are provided herein.

In at least one embodiment, a composite stent (having a length and a circumference) comprises an expandable framework and at least one metallic structure. The expandable framework defines a plurality of openings, each opening having a perimeter defined by a plurality of struts of the expandable framework. Each strut has an outer surface, an inner surface, and a radial surface between the outer surface and the inner surface. The at least one metallic structure spans across the opening to connect the lateral surface of a first strut to the lateral surface of a second strut on an opposite side of the opening.

### BRIEF DESCRIPTION OF THE DRAWING(S)

FIG. 1 is a perspective view of a composite stent of the present invention.

FIG. 2 is an enlarged view of a portion of an embodiment of the composite stent shown in FIG. 1.

FIG. 3 is an enlarged view of a portion of an embodiment of the composite stent shown in FIG. 1.

FIG. 4 is an enlarged view of a portion of an embodiment of a mold for manufacturing the composite stent of FIG. 1

FIG. 5A is an enlarged view of a portion of one embodiment of a composite stent of the present invention.

FIG. 5B is a cross-section of a strut of the composite stent shown in FIG. 5A.

FIG. 6 is an enlarged view of a portion of one embodiment of a composite stent of the present invention.

### DETAILED DESCRIPTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

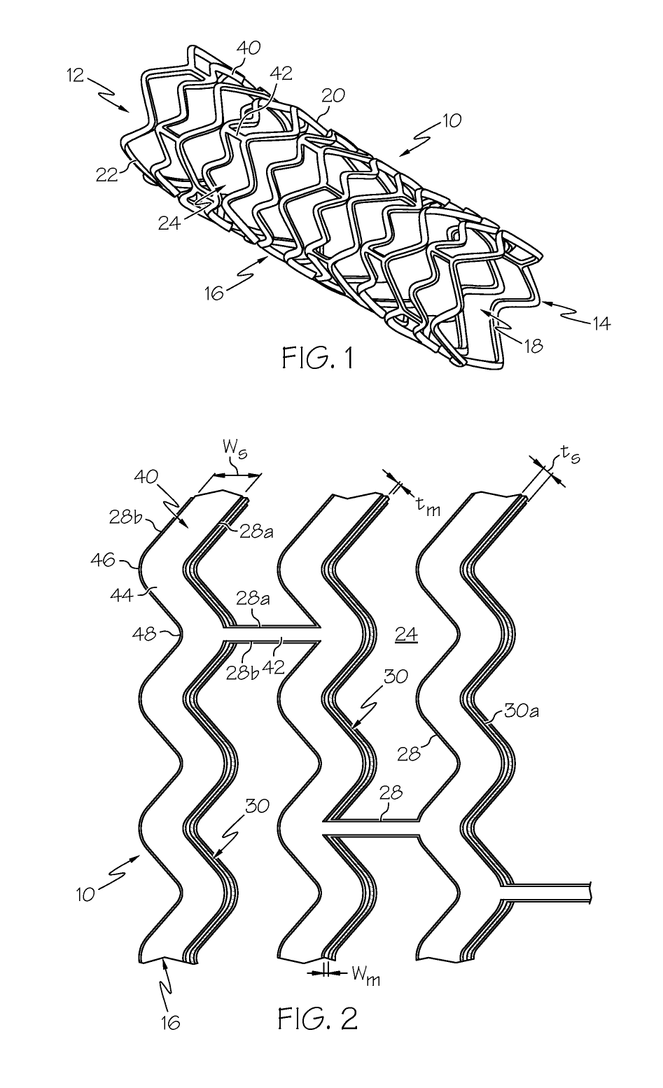

FIG. 1 shows one embodiment of a composite stent of the present invention in an expanded state, and FIG. 2 shows an enlarged view of the embodiment of the composite stent shown in FIG. 1. The composite stent 10 comprises a first end 12, a second end 14, and an expandable framework 16 disposed about a longitudinal axis of the stent that defines a lumen 18 therethrough. The expandable framework 16 is expandable from an unexpanded state to the expanded state shown in FIG. 1. The expandable framework 16 has an outer surface 20 and an inner surface 22. In at least one embodiment, the outer surface 20 is the abluminal surface of the composite stent, and the inner surface 22 is the luminal surface of the composite stent. The expandable framework 16 has a thickness between the outer surface 20 and the inner surface 22. In at least one embodiment, the expandable framework 16 comprises a bioabsorbable polymer, such as poly-L-lactide (PLLA), polyglycolide (PGA), polylactide, (PLA), poly-D-lactide (PDLA), polycaprolactone, polydioxanone, polygluconate, polylactic acid-polyethylene oxide copolymers, modified cellulose, collagen, poly(hydroxybutyrate), polyanhydride, polyphosphoester, poly(amino acids), and combinations thereof.

The expandable framework 16 defines a plurality of openings 24. Each opening 24 has a perimeter defined by radial surfaces (or side walls) 28 of the expandable framework. Each radial surface 28 extends between the outer surface 20 and the inner surface 22. In at least one embodiment (shown better in FIG. 2), at least one metallic structure 30 is disposed onto at least one radial surface 28 of the expandable framework 16. In at least one embodiment, the metallic structure 30 is a stiffener. In at least one embodiment, the metallic structure 30 comprises a bioabsorbable metal, such as iron, iron alloys, magnesium, magnesium alloys, or a metal that is not bioabsorbable, such as cobalt, cobalt alloys such as L605, stainless steel alloys such as 316L, nickel, nickel alloys such as MP35N and Elgiloy, titanium, titanium alloys such as NiTi and Ti6Al4V, tantalum, niobium, tungsten, gold, platinum, iridium, palladium, molybdenum, zirconium, and alloys thereof those elements. The metallic structure 30 provides additional radial strength and stiffness to the expandable framework, and in some embodiments provides radioopacity for the composite stent. In at least one embodiment, there is no metal on the outer surface 20 and there is no metal on the inner surface 22 of the expandable framework 16. Any metal coating on the expandable structure (such as the metallic structure 30) is only on the radial surface or sidewall of the strut that defines the perimeter of the opening.

In at least one embodiment, each opening 24 of the expandable stent has a metallic structure 30 disposed on the radial surface 28. In at least one embodiment, only some of the openings 24 of the expandable stent have a metallic structure 30 disposed on the radial surface 28.

In one embodiment, the openings at the ends of the stent could have a metallic structure to enhance strength and stiffness there and avoid constriction of the fluid flow inlet and outlet of the stented vessel. In one embodiment, the openings comprising one-half the overall length of the stent centered about the stent length mid-point could have a metallic structure, so that stent strength and stiffness are greatest where the stent overlaps the lesion and are less where the stent overlaps healthier vessel tissue that does not need so much scaffolding support. In one embodiment, the openings in one-half of the stent length from mid-stent length to one end could have a metallic structure such that the stent could be implanted in ostial lesions and the portion of the stent extending out into the ostium (not in contact with vessel wall) would not have metallic structures that could be liberated from the stent and enter into systemic circulation. In one embodiment, the openings along the length of the stent within a circumferential arc of 10 to 180 degrees could have a metallic structure which could be oriented during implantation to be adjoined to an eccentric lesion.

In one embodiment, the metallic structures 30 are disposed within openings 24 at specific locations to increase stiffness, strength, and radioopacity at desired locations along the stent. In at least one embodiment, openings 24 with a metallic structure 30 alternate axially along the length of the composite stent 10 with openings 24 that do not have a metallic structure disposed on the radial surface 28. In at least one embodiment, openings 24 with a metallic structure 30 alternate radially along a circumference of the composite stent 10 with openings 24 that do not have a metallic structure disposed on the radial surface 28.

In the at least one embodiment, the first metallic structure 30 covers at least a portion of the perimeter of the opening 24 in both the expanded state and the unexpanded state. In at least one embodiment, the first metallic structure covers the entire perimeter of the opening 24 in both the expanded state and the unexpanded state. In some embodiments, the first metallic structure 30 can be a layer of material, a strut, or a ring-like form. Where the metallic structure 30 is ring-like, the metallic structure has an outer perimeter that is at least substantially similar, if not equivalent, to the perimeter of the opening. The outer perimeter is substantially similar if it is about the same size and shape as the perimeter of the opening 24. In any of the embodiments, the metallic structure 30 can be adhered to the radial surface 28. In at least the embodiment where the metallic structure 30 is a ring-like form, the metallic structure 30 is force fit into the opening 24 of the expandable framework 16.

While the expandable framework 16 can have any configuration, in some embodiments (such as the embodiment shown in FIG. 1), the expandable framework 16 comprises a plurality of axially adjacent circumferential bands 40. In at least one embodiment, each circumferential band 40 is connected to an axially adjacent circumferential band 40 by a connector strut 42. In at least the embodiment shown, each circumferential band 40 has a serpentine configuration comprising a plurality of struts 44 forming a plurality of alternating peaks 46 and troughs 48. In other embodiments, the circumferential band 40 can be formed of struts 44 with other configurations.

In at least the embodiment shown in FIGS. 1 & 2, struts 44 and connector struts 42 define each opening 24. In at least one embodiment, the first metallic structure 30 is disposed on struts 44 and connector struts 42 that define an opening 24.

As shown, composite stent 10 comprises the expandable framework 16 and the metallic structure 30. As discussed above the expandable framework 16 has a plurality of struts 42, 44 that define a plurality of openings 24. The struts 42, 44 of the expandable framework 16 each have a width, Wₛ, and a thickness tₛ, where the thickness tₛ is defined between the outer surface 20 and the inner surface 22 of the expandable framework 16. The struts 42, 44 each have a first radial surface 28a, and a second radial surface 28b. As shown in FIG. 2, the first metallic structure 30 is disposed onto at least a first radial surface 28a of at least one of the struts 42, 44.

In at least one embodiment, the metallic structure 30 also has a width, Wₘ, and a thickness tₘ. In the embodiment shown the width, Wₘ, of the metallic structure 30 is in the same direction as the width, Wₛ, of the strut 42, 44. Likewise, the thickness, tₘ, of the metallic structure 30 is in the same direction as the width, tₛ, of the strut 42, 44. In some embodiments, the metallic structure 30 has a width, Wₘ, less than the width, Wₛ, of the strut 42, 44 on which the metallic structure 30 is disposed, adhered to, or otherwise joined. In at least one embodiment, the width Wₘ of the metallic structure 30 is at least one order of magnitude less than the width Wₛ of the strut 42, 44. In at least one embodiment, the width Wₘ of the metallic structure 30 is between about 5% and 50% of the width Wₛ of the strut 42, 44.

In at least one embodiment, the width Wₘ of the metallic structure 30 is between about 40% and 50% of the width Wₛ of the strut 42, 44. In at least one embodiment, the width Wₘ of the metallic structure 30 is between about 30% and 40% of the width Wₛ of the strut 42, 44. In at least one embodiment, the width Wₘ of the metallic structure 30 is between about 20% and 30% of the width Wₛ of the strut 42, 44. In at least one embodiment, the width Wₘ of the metallic structure 30 is between about 10% and 20% of the width Wₛ of the strut 42, 44. In at least one embodiment, the width Wₘ of the metallic structure 30 is between about 5% and 10% of the width Wₛ of the strut 42, 44.

In at least one embodiment, the metallic structure 30 has a thickness tₘ that is equal to the thickness tₛ of the strut 42, 44 on which the metallic structure 30 is disposed, adhered to, or otherwise joined. In at least one embodiment, the metallic structure 30 has a thickness tₘ that is less than the thickness tₛ of the strut 42, 44 on which the metallic structure 30 is disposed. In at least one embodiment, the thickness tₘ should be much less than the strut thickness so as to minimize volume of metal in the stent and have a substantially bioabsorbable polymer stent.

In at least one embodiment, the thickness tₘ of the metallic structure 30 is less than about 90% of the thickness tₛ of the strut 42, 44. In at least one embodiment, the thickness tₘ of the metallic structure 30 is between about 80% and 90% of the thickness tₛ of the strut 42, 44. In at least one embodiment, the thickness tₘ of the metallic structure 30 is between about 70% and 80% of the thickness tₛ of the strut 42, 44. In at least one embodiment, the thickness tₘ of the metallic structure 30 is between about 60% and 70% of the thickness tₛ of the strut 42, 44. In at least one embodiment, the thickness tₘ of the metallic structure 30 is between about 50% and 60% of the thickness tₛ of the strut 42, 44. In at least one embodiment, the thickness tₘ of the metallic structure 30 is between about 40% and 50% of the thickness tₛ of the strut 42, 44. In at least one embodiment, the thickness tₘ of the metallic structure 30 is between about 30% and 40% of the thickness tₛ of the strut 42, 44. In at least one embodiment, the thickness tₘ of the metallic structure 30 is between about 20% and 30% of the thickness tₛ of the strut 42, 44. In at least one embodiment, the thickness tₘ of the metallic structure 30 is between about 10% and 20% of the thickness tₛ of the strut 42, 44. In at least one embodiment, the thickness tₘ of the metallic structure 30 is less than 10% of the thickness tₛ of the strut 42, 44. In at least one embodiment, the thickness tₘ of the metallic structure 30 is less than 5% of the thickness tₛ of the strut 42, 44. In at least one embodiment, the thickness tₘ of the metallic structure 30 is between about 1% and 5% of the thickness tₛ of the strut 42, 44.

The width Wₘ and thickness tₘ of the metallic structure 30 is dependent upon the material used for the metallic structure and the desired increase in strength or stiffening of the expandable framework.

The composite stent 10 has different material work hardening rates between the expandable framework 16 and the metallic structure 30. Plastic deformation establishes the shape of the expanded stent within the vessel. If only elastic deformation occurred in the material, the expanded stent would recoil or spring back to near the original as-manufactured shape upon release of balloon pressure. Importantly, as the composite stent 10 is plastically deformed during expansion, portions of the composite stent 10 that have high strain undergo strain work hardening due to an increase in dislocation density in metals or molecular chain orientation changes in polymers. Strain work hardening increases yield strength, which increases radial strength of the composite stent 10 relative to the expandable framework 16 alone. The metallic structures 30 present additional work hardening to the stent construction. The composite stent 10 has higher stent radial and hoop strength and lower recoil relative to the expandable framework alone—at least 25% higher radial strength and at least 25% less elastic recoil upon expansion of the composite stent. This means that the polymer stent need not be significantly (at least 30%) thicker than a metal stent of the same design in order to have comparable strength and stiffness without compromising the stent delivery system profile. The polymer stent with metal strut wall lining would have at least 50% less metal volume than a comparable, single-material metal bioabsorbable stent. For example, a composite stent of the present invention having metallic structures that are each 25% of the width of the overall strut width would have 50% less metal volume than a single material metal stent of the same design. A polymer stent with metal strut wall lining components that are each one-eight of the width of the overall strut width would have 75% less metal volume than a single-material metal stent of the same design.

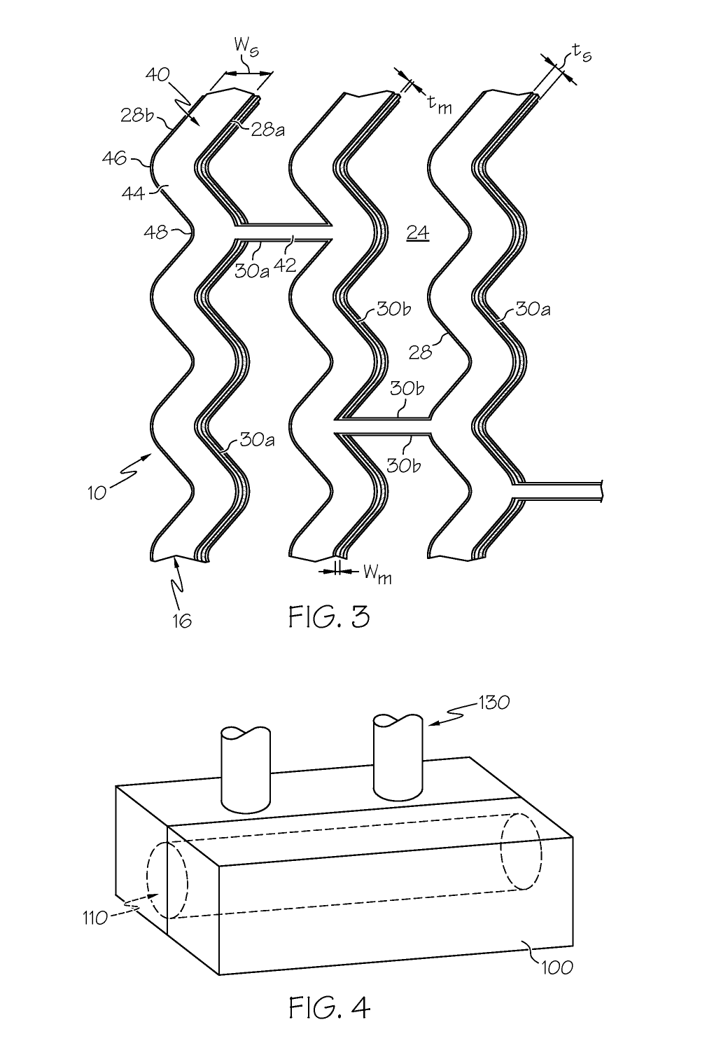

In at least one embodiment, shown in FIG. 3, a first metallic structure of a first material 30a alternates axially with a second metallic structure of a second material 30b along the length of the composite stent 10. In one embodiment, the first metallic structure 30a has the same width and the same thickness as the second metallic structure 30b. In one embodiment, the first material is a bioabsorbable metal and the second material is a radiopaque metal. For example, the first material can be iron and the second material can be L605. While the iron is degradable, the L605 is not bioabsorbable. Rather, upon complete degradation of the polymer, the vessel vasomotion is favorably returned to a more natural condition (relative to a vessel with a permanent metal stent) because there is no longer an interconnected network of polymer or metal. There is a minimal volume of permanent metal remaining in the stented lumen after stent bioabsorption relative to a permanent metal stent implant.

In at least one embodiment, a first metallic structure of a first material 30a alternates radially with a second metallic structure of a second material 30b about the circumference of the composite stent 10.

To manufacture one of the composite stents 10 described above and shown in the figures, various methods can be utilized. One exemplary method is to laser cut the expandable framework 16 from a tube of a bioabsorbable polymer, such as poly-L-lactic acid polymer (PLLA). In some embodiments, the as-cut framework is then cleaned to remove laser machining debris. The metallic structure 30 is then adhered to or force fit into an opening 24 of the expandable framework 16.

The metallic structure 30 can be adhered to the expandable framework 16 with a cyanoacrylate adhesive, an adhesive made of a bioabsorbable polymer such as PLA. The metallic structure 30 can be attached to the expandable framework 16 by overcoating the metallic structure 30 while positioned within the stent opening such that the metallic structure 30 and the strut 42 are encapsulated by a coating of bioabsorbable polymer. The bioabsorbable polymer can be put into solution and sprayed onto the assembly or the assembly could be dipped or roll coated in the polymer solution. The radial surface 28 of the expandable framework 16 could be beveled or channeled with injection molding, mechanical micromachining, chemical machining, or laser machining techniques to create a groove in the radial surface 28 such that the metal is pressed or deposited into the groove. Force fitting or pressing the metallic structure 30 onto the radial surface 28 can be performed while the expandable framework 16 is heated to a temperature that softens the polymer and allows it to flow and partially or fully envelope the metallic structure 30. Pressing or force fitting the metallic structure 30 against the polymer radial surface 28 without heating can be done such that there is a slight interference fit between the metallic structure and the struts forming the perimeter of the opening, such that the metallic structure will not fall out of the stent. The interference fit could be designed to not exceed the elastic limit or plastic limit of the bioabsorbable polymer of the expandable framework 16 in order to avoid fracture of the expandable framework 16. In at least one embodiment, metal is directly applied to the radial surface 28 to form the metallic structure 30.

In another exemplary method, shown in FIG. 4, a mold 100 is provided having a cavity 110 for a mold insert fixture (not shown) and at least one injection port 130. The mold insert fixture has a pattern for the shape of the expandable framework. A plurality of metallic structures 30 are held onto the mold insert fixture. The mold insert fixture is then positioned into the mold cavity 110. A polymer resin, such as PLLA polymer, is injected into the mold cavity 110 through the at least one injection port 130 to form the expandable framework 16. Thus, in this embodiment, the metallic structures 30 are integrally formed with the expandable framework 16, rather than adhered or force fit into an as-cut framework.

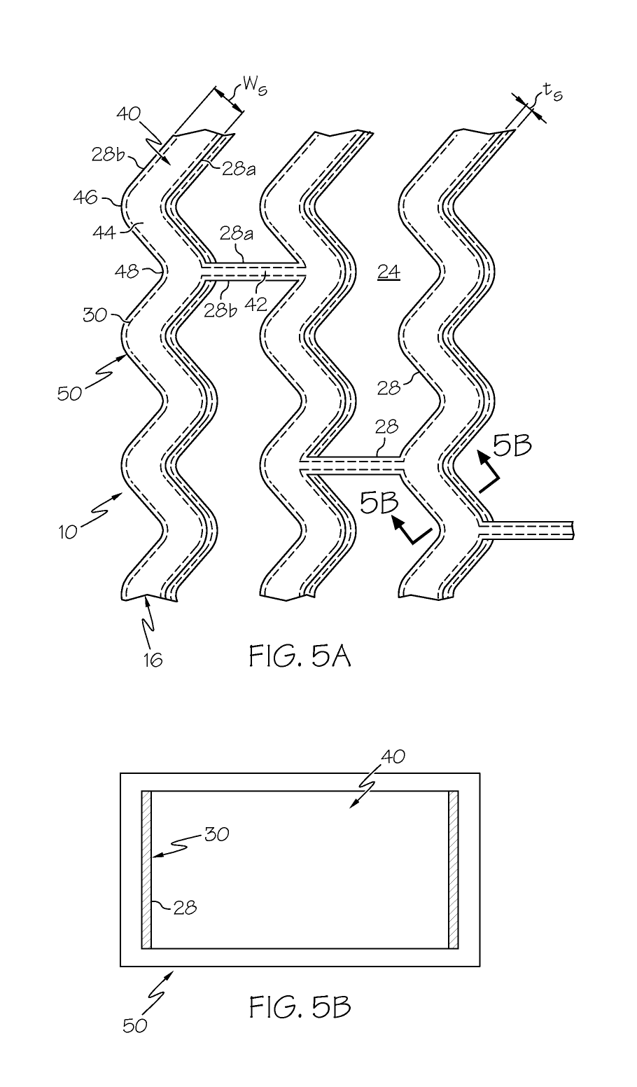

FIG. 5A shows another embodiment of a composite stent 10 in an expanded state, and FIG. 5B shows a cross-section of the composite stent 10 having an outer surface 20 and an inner surface 22. The composite stent 10 comprises an expandable framework 16. The expandable framework 16 is expandable from an unexpanded state to the expanded state. In at least one embodiment, the expandable framework 16 comprises a bioabsorbable polymer, such as poly-L-lactide (PLLA), polyglycolide (PGA), polylactide, (PLA), poly-D-lactide (PDLA), polycaprolactone, polydioxanone, polygluconate, polylactic acid-polyethylene oxide copolymers, modified cellulose, collagen, poly(hydroxybutyrate), polyanhydride, polyphosphoester, poly(amino acids), and combinations thereof.

While the expandable framework can have any configuration, in some embodiments (such as the embodiment shown in FIG. 5A), the expandable framework 16 comprises a plurality of axially adjacent circumferential bands 40. In at least one embodiment, each circumferential band 40 is connected to an axially adjacent circumferential band 40 by a connector strut 42. In at least the embodiment shown, each circumferential band 40 has a serpentine configuration comprising a plurality of struts 44 forming a plurality of alternating peaks 46 and troughs 48. In other embodiments, the circumferential band 40 can be formed of struts 44 with other configurations. In at least the embodiment shown in FIGS. 1 & 2, struts 44 and connector struts 42 each have radial surfaces 28. Each radial surface 28 extends between the outer surface 20 and the inner surface 22.

In at least the embodiment shown, a plurality of metallic structures 30 are disposed onto the radial surfaces 28 of the expandable framework 16. In at least the embodiment shown, the metallic structures 30 are strut-like members. In one embodiment (as shown in FIG. 5A), each metallic structure 30 has a length that is less than the length of the strut on which the metallic structure is disposed. In one embodiment, the metallic structures 30 are each spaced apart along the radial surfaces 28 of the expandable framework 16. In at least the embodiment shown, a coating layer of polymeric material 50 is applied to the outer surfaces of the both the metallic structures 30 and the expandable framework 16. In at least one embodiment, the coating layer 50 encapsulates the metallic structures 30 and the expandable framework 16, preventing the metallic structures from separating from the expandable framework during crimping of the composite stent onto the delivery system and upon implantation.

In at least one embodiment, each opening 24 of the expandable framework 16 has a metallic structure 30 disposed on the radial surface 28. In at least one embodiment, only some of the openings 24 of the expandable framework 16 have a metallic structure 30 disposed on the radial surface 28. In at least one embodiment, openings 24 with a metallic structure 30 alternate axially along the length of the composite stent 10 with openings 24 that do not have a metallic structure disposed on the radial surface 28. In at least one embodiment, openings 24 with a metallic structure 30 alternate radially along a circumference of the composite stent 10 with openings 24 that do not have a metallic structure disposed on the radial surface 28. In one embodiment, the metallic structures 30 are disposed within openings 24 at specific locations to increase stiffness, strength, and radioopacity at desired locations along the stent.

To manufacture the composite stent 10 shown in FIGS. 5A-5B described above, various methods can be utilized. One exemplary method is to laser cut the expandable framework 16 from a tube of a bioabsorbable polymer, such as poly-L-lactic acid polymer (PLLA). In some embodiments, the as-cut framework is then cleaned to remove laser machining debris. The outer surface and the inner surface of the expandable framework 16 can be masked with a lacquer. In at least one embodiment, metal is cold vapor deposited onto the radial surfaces 28 to form the metallic structures 30. The lacquer, if applied, is then removed by soaking the composite stent 10 in acetone. The expandable framework 16 with the metallic structures 30 is then dip coated with a polymer material such as PLLA to fully encapsulate the expandable framework 16 and the metallic structure 30.

In another exemplary method, the metallic structures 30 are adhered to the radial surfaces 28 of the expandable framework 16. The expandable framework 16 with the metallic structures 30 is then dip coated with a polymer material such as PLLA to fully encapsulate the expandable framework and the metallic structure.

In another exemplary method, a mold 100, such as the one shown in FIG. 4, is provided having a cavity 110 for a mold insert fixture (not shown) and at least one injection port 130. In one embodiment, the expandable framework 16, with the metallic structures 30 adhered to the expandable framework 16 or cold vapor deposited onto the expandable framework 16, is held onto the mold insert fixture. The mold insert fixture is positioned into the mold cavity 110. A polymer resin, such as PLLA polymer, is injected into the mold cavity 110 through the at least one injection port 130 to form the coating layer 50.

In another exemplary method, the metallic structures 30 are held onto the mold insert fixture, which has a pattern for the expandable framework 16. The mold insert fixture is positioned into the mold cavity 110. A polymer resin, such as PLLA polymer, is injected into the mold cavity 110 through the at least one injection port 130 to form both the coating layer 50 and the expandable framework 16 simultaneously. The expandable framework 16 and metallic structures 30 can then be dip coated with a polymer material such as PLLA to fully encapsulate the expandable framework and the metallic structure. Alternatively, a first injection of a first polymer resin can be used to form the expandable framework 16 and a second injection of a second polymer resin can be used to form the coating layer 50. In one embodiment, the same mold can be used for both injection steps. In another embodiment, the mold insert fixture may be transferred from a first mold to a second mold between the first injection and the second injection.

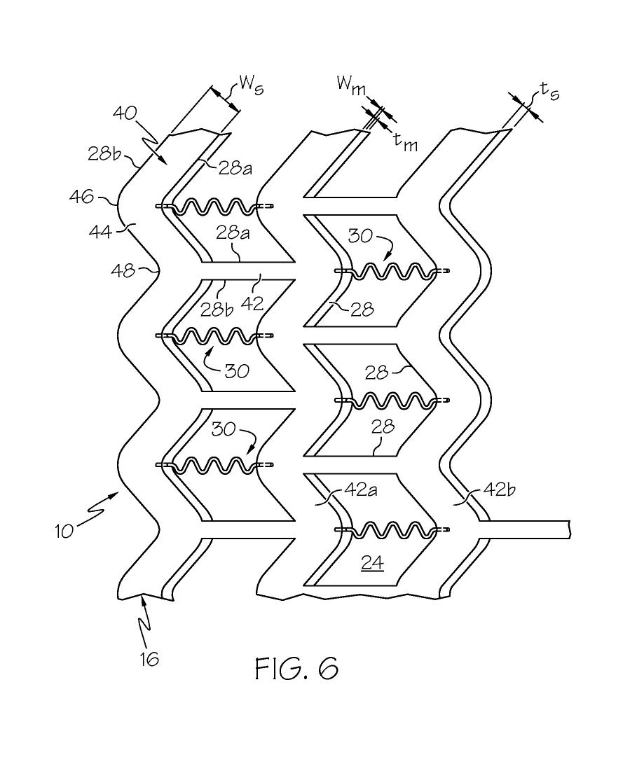

FIG. 6 shows another embodiment of composite stent 10. In this embodiment, the metallic structure 30 is a strut that connects one strut 42,44 with another strut 42,44 of the expandable framework 16. In at least one embodiment, the metallic structure connects a radial surface 28 of one strut 42, 44 to a radial surface 28 of another strut 42, 44. In at least one embodiment, the metallic structure 30 connects a first strut 42a with a second strut 42b directly opposite the first strut, or directly across the opening 24. In at least one embodiment the metallic structure 30 spans the opening 24. The metallic structure 30 can have any configuration, including but not limited to a straight configuration, a zig-zagged configuration, a helical or sinusoidal coiled configuration, looped (for example, eyelooped), and combinations thereof. In at least one embodiment, the radial surface 28 where the metallic structure is joined to the strut 42, 44 with a polymer material to enclose the ends of the metallic structure 30 within the polymer strut. In at least one embodiment, this polymer material is the same material as the expandable framework 16. In at least one embodiment each metallic structure has a width that is at least half the width of the strut. In at least one embodiment, each metallic structure has a thickness that is half the thickness of the strut. In at least one embodiment the width and the thickness of the metallic structure are the same.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction. In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A composite stent having a length and a circumference, the composite stent comprising:
an expandable framework comprising a bioabsorbable polymer, the expandable framework defining a plurality of openings, each opening having a perimeter defined by a plurality of struts of the expandable framework, wherein each strut has an outer surface, an inner surface and a thickness between the outer surface and the inner surface; and
at least one first metallic structure disposed only along at least a portion of the perimeter of at least one of the openings, the first metallic structure comprising a first material, the first metallic structure having a width and a thickness.

2. The composite stent of claim 1, wherein the thickness of the at least one first metallic structure is less than the thickness of the strut.

3. The composite stent of claim 1, wherein each strut has a width, and the width of the at least one first metallic structure is less than the width of the strut.

4. The composite stent of claim 1, wherein the first material is a metal selected from the group consisting of iron, iron alloys, cobalt, cobalt alloys, magnesium, magnesium alloys, stainless steel alloys, nickel, nickel alloys, titanium, titanium alloys, tantalum, niobium, tungsten, gold, platinum, iridium, palladium, molybdenum, zirconium, and combinations thereof.

5. The composite stent of claim 4, wherein the bioabsorbable polymer is selected from the group consisting of poly-L-lactide (PLLA), polyglycolide (PGA), polylactide, (PLA), poly-D-lactide (PDLA), polycaprolactone, polydioxanone, polygluconate, polylactic acid-polyethylene oxide copolymers, modified cellulose, collagen, poly(hydroxybutyrate), polyanhydride, polyphosphoester, poly(amino acids), and combinations thereof.

6. The composite stent of claim 1, wherein the at least one first metallic structure is adhered to the adjacent strut with an adhesive.

7. The composite stent of claim 1, wherein the at least one first metallic structure is a ring-like structure.

8. The composite stent of claim 1, wherein the at least one first metallic structure is force fit into the opening of the expandable framework.

9. The composite stent of claim 1, further comprising at least one second metallic structure disposed along at least a portion of the perimeter of at least one of the openings, the at least one second metallic layer comprising a second material different than the first material.

10. The composite stent of claim 9, wherein first metallic structures alternate radially with second metallic structures along the circumference of the composite stent.

11. The composite stent of claim 9, wherein first metallic structures alternate axially with second metallic structures along the length of the composite stent.

12. The composite stent of claim 1, further comprising a coating layer of polymeric material encapsulating the at least one first metallic structure and the expandable framework, wherein the coating layer comprises a polymeric material.

13. A composite stent having a length and a circumference, the composite stent comprising:
a bioabsorbable polymer framework that is expandable, the framework defining a plurality of openings, each opening having a perimeter defined by a plurality of polymer struts of the framework, wherein each polymer strut has an outer surface, an inner surface, and a radial surface between the outer surface and the inner surface; and
at least one first metallic structure spans across the opening and connects the radial surface of a first strut to a radial surface of the second strut on an opposite side of the opening, wherein the first metallic structure comprising a first material.

14. The composite stent of claim 13, the at least one metallic structure has a configuration selected from the group consisting of straight configurations, zig-zagged configurations, coiled configurations and combinations thereof.

15. The composite stent of claim 13, wherein the first material is a bioabsorbable metal selected from the group consisting of iron, iron alloys, cobalt, cobalt alloys, magnesium, magnesium alloys, and combinations thereof.

16. A method for manufacturing a composite stent comprising:
positioning a mold insert fixture into a cavity of a mold, wherein the mold insert fixture has a plurality of metallic structures and a pattern for the shape of an expandable network;
injecting a polymer resin into the mold cavity to integrally form the expandable framework with the metallic structures, wherein the polymer resin is injected into the mold cavity through an injection port of the mold.

17. The method of claim 16, further comprising forming a coating layer with a polymer material to fully encapsulate the expandable framework and the metallic structures.
